(19) United States
(12) Patent Application Publication
(10) Pub. No.: US 2026/0256987 A1
(43) Pub. Date: Sep. 3, 2026

(54) MANUFACTURE OF ADIPOSE-DERIVED HUMAN ADULT MESENCHYMAL STROMAL CELLS

(71) Applicant: Theracell Laboratories, Kifissia, Attika (GR)

(72) Inventors: FOTIOS SAKELLARIDIS, Kifissia, Attika (GR); POLYCHRONIS FATOUROS, Kifissia, Attika (GR); MARIA KONTOU, Kifissia, Attika (GR)

(73) Assignees: Theracell Laboratories, 27 Evrota Street (GR); Orgenesis Inc., Germantown, MD (US)

(21) Appl. No.: 18/867,281

(22) PCT Filed: May 17, 2023
(86) PCT No.: PCT/US23/67154

§ 371 Date: Nov. 19, 2024

Related U.S. Application Data
(60) Provisional application No. 63/343,831, filed on May 19, 2022.

(51) Int. Cl. A61L 27/38; C12N 5/077

(52) U.S. Cl. CPC A61L 27/3834; C12N 5/0669; A61L 2400/06; A61L 2430/24

(57) ABSTRACT

Disclosed herein is an autologous composition comprising mesenchymal stem stromal cells, wherein the mesenchymal stem stromal cells are derived from adipose tissue collected by punch biopsy from a subject, and the use for the treatment of a disease, including a disease with arthritic lesion progression.

### BACKGROUND OF THE INVENTION

Human mesenchymal stem cells or mesenchymal stromal cells (hMSCs) are commonly tested in adult stem cells in therapy. It is now apparent that hMSCs empower tissue repair abilities by secretion of immune-regulatory and pro-regenerative factors. A growing trend in hMSCs clinical trials is the use of allogenic or autologous, culture-expanded cells. Unfortunately, the clinical outcomes using manufactured hMSCs have been inconsistent and suboptimal. Donor co-morbidity, cryopreservation, large-scale expansion and inappropriate delivery roots and dosage are known to alter hMSCs profile and reduce therapeutic potency, which are significant barriers in hMSCs clinical translation. Each of these factors, and probably others, currently unknown factors, can reduce hMSCs immunomodulatory capacity and, therefore, reduce their therapeutic potency.

Among the core pathways to improving hMSCs function, metabolism has emerged as an important hub. hMSCs are heterogeneous at the level of primary metabolism and their energy metabolism status plays important roles in regulating hMSCs functional properties and effector functions.

Current hMSC manufacturing practices lead to metabolic shift that reduces therapeutic properties. hMSC are utilized freshly isolated and expanded under artificial environment to obtain sufficient cell number for clinical application. However, external stress during isolation and expansion shifts hMSC metabolic state from steady-state to stressed phenotype which increases senescent subset and contributed to a breakdown of cellular homeostasis.

Increased reactive oxygen species (ROS) inhibit MSC proliferation, increase senescence, promote apoptotic cell death and enhance adipogenic but reduce osteogenic differentiation and inhibit MSC immunomodulation. Oxidative stress affects ex vivo culture expansion and longevity of MSCs, which has implications for cell therapy success.

It was shown that MSCs from older donors, which also are likely to have greater oxidative stress, have reduced capacity to inhibit T cell proliferation.

There is still a need for metabolic preconditioning targeting specific pathways that can restore hMSC cellular homeostasis and enhance their therapeutic potency.

### SUMMARY OF THE INVENTION

In some aspects, disclosed herein is an autologous composition comprising mesenchymal stromal cells, wherein the mesenchymal stromal cells are derived from adipose tissue collected by punch biopsy from a subject. In one embodiment, the cells are preserved in their natural anatomic compartment and tissue architecture is preserved up until isolation manipulations take place. In one embodiment, the cells are embedded in fat tissue in the hypoxic state that is needed for their optimal metabolic activity, up until isolation manipulation take place.

In some related aspects, the composition comprises of 10-100×10⁶ mesenchymal stromal cells resuspended in buffered saline or other appropriate final resuspension medium. In some further related aspects, the composition comprises of 20-25×10⁶ mesenchymal stromal cells resuspended in buffered saline or other appropriate final resuspension medium.

In some related aspects, the suspension medium presents reduced levels of Lactate Dehydrogenase (LDH) activity, reduced reactive oxygen species or combination thereof.

In some related aspects, the disclosed herein is the use of the composition for treating a disease in the subject. In some further related aspects, the disease comprises arthritis lesion progression. In some further related aspects, the disease comprises osteoarthritis.

In some aspects, disclosed herein is a method for the preparation of a composition comprising autologous mesenchymal stromal cells, comprising the step of collecting adipose tissue by punch biopsy. In some further aspects, the tissue biopsy ranges from 0.5-2 g of tissue. In some further aspects, the method further comprises the steps of a. Isolating and culturing the adipose-derived stromal cells; b. Collecting the adipose-derived stromal cells; c. Expanding the mesenchymal stromal cells ex vivo in a Grade B clean room facility under GMP operation; and c. Cryopreserving and thawing of cryopreserved adipose-derived stromal cells.

In some aspects, disclosed herein is a method for delaying osteoarthritic lesion progression, inducing repair and regeneration of inflamed/destructed tissue, or combination thereof, in a subject, comprising administering an autologous composition comprising mesenchymal stromal cells, wherein said mesenchymal stromal cells are derived from adipose tissue collected by punch biopsy from a subject, wherein said subject in need of delaying osteoarthritic lesion progression, inducing repair and regeneration of inflamed/destructed tissue or any combination thereof.

In some further aspects, the subject is diagnosed with osteoarthritis, multiple sclerosis, amyotrophic lateral sclerosis, degenerative disc disease, traumatic spinal cord injury, cardiovascular disease, atherosclerosis, ischemic myocardial tissue, chronic kidney disease, skin lesions, burns, ulceration, inflammatory lung disease or multiple causes or any combination thereof. In some further aspects, the autologous composition is administered intra-articularly, systemically, intra-vascularly, intra-cerebroventricularly, sub-cutaneously, intra-dermally, intrathecally or intra-myocardially.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a graphic illustration of the presence of Lactate Dehydrogenase (LDH). More LDH is detected in the Lipoaspirate (LA) sample compared to the Fat Biopsy (FB) sample, both from the same subject. The values for FB have been normalized to 1 as reference, values from LA are ~3-fold higher. Dual samples from 2 different patients have been used for these data (N=2). Error bar represents standard deviation (SD);

FIG. 2 is a graphic illustration of reactive oxygen species (ROS) generation in crude cell mix isolated from FB and LA samples. Higher relative values for mean fluorescence intensity are detected for the LA samples, 1.5 times up to 2-fold increase. N=2 and error bars represent SD;

FIGS. 3A and 3B illustrate correlation plots of the weight of processed adipose tissue biopsy (X axis) to the days needed to complete Passage 0 (Y axis) The color of the bubbles represents the cell numbers according to the heat scale on the right and the size of the bubbles is proportional to the number of cells to help visualization. Panel A is for the GMP-process (Nutristem+5% human platelet lysate (hPL)) and panel B corresponds to the R&D process (DMEM+10% FBS);

FIG. 4 is a graphic illustration of inhibitory effects of MSCs on stimulated PBMC proliferation. PBMCs were cultured alone or cocultured with naïve or licensed MSCs under Transwell condition for 7 days. Afterward, cell numbers were counted using a Cell Counting Kit-8. Graphs showing the cell proliferation relative to unstimulated PBMCs. Data are presented as the mean±SD. Each data point represents one sample. Comparisons by ANOVA with Dunnett's multiple comparisons test;

FIG. 5 presents histograms of the fluorescent intensities (Arbitrary Units) from a representative example of the same batch of cells in unstimulated (left panel, light gray) and stimulated/licensed state (right panel, dark gray). The more intense fluorescence in the 2^{nd} case is evident by the clear increase in the “Area Under the Curve” observed for the licensed MSCs;

FIG. 6 presents the raw fluorescence intensity measured with the Arthur fluorescent Cell Counter from unstimulated cells (left panel) and licensed MSCs (right panel) that were from the same cell batch prior to stimulation. The gray vertical line is the Relative Fluorescence Unit Threshold applied. It is evident that the vast majority of measurements from the unstimulated control sample lie on the left side of the threshold (are lower) while a prominent shift to greater fluorescence values and a move of the curve to the right side of the thresholds took place in the read-out from the licensed MSCs;

FIG. 7 represents Time course quantification of the percentage of IDO+ cells from 4 independent AD-MSC samples, at 4 different time points: time 0 (unstimulated, control) 4 hours post-stimulation, 24 hrs post-stimulation and 48 hrs post-stimulation. While 4 hrs is not adequate to show a robust increase in the signal for the intracellular enzyme IDO, at 24 hrs the distinction is clear, robust and reproducible;

FIG. 8A represents AD-MSCs embedded in the Tisseel scaffold minutes after seeding and addition of medium (Day 0). The amorphous mass of the clot is visible, peppered with round cells. 10× objective, Leica DM LB. FIG. 8B represents diffraction image of the boundary between the fibrin glue clot and the well surface. Rounded cells are seen inside the amorphous clot and at the boundary, cells are migrating out, acquiring the typical in vitro morphology of AD-MSCs, atractoid with several processes. 10× objective, Leica DM LB. FIG. 8C represents AD-MSCs in fibrin glue, 1 day post-seeding, maintaining their amoeba-like shape, unlike the cells that grow on plastic. 40× objective, Leica DM LB. FIG. 8D represents a depiction of the edge of a fibrin glue clot (left/top-left) at the border with the plastic surface which is covered with cells (bottom and right part of picture). At the middle of the picture a “peninsula” of the clot is evident from which the cells seem to have spread out and onto the surface, populating it with a dense mesh of interdigitating cells;

FIG. 9 is a stereoscope view of a well with Tisseel fibrin glue clot stained with Alizarin Red, depicting osteogenesis. Hubs of positively stained red calcium deposits are also observed peppered across the rest of the well surface, with more intense color inside and around the scaffold.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Autologous Composition.

In some embodiments, disclosed herein is an autologous composition comprising mesenchymal stromal cells, wherein said mesenchymal stromal cells are derived from adipose tissue collected by punch biopsy from a subject.

Mesenchymal stromal cells (MSCs) are pluripotent progenitor cells that contribute to the maintenance and regeneration of diverse tissues. MSCs can be found in many tissues where they serve as local sources of stromal cells, such as bone marrow, blood or different sources of mesenchymal tissue. MSCs contribute to tissue remodeling after injury or during chronic inflammation. The damaged tissue is thought to release specific endocrine cues that then lead to the mobilization of multi-potent MSCs and their subsequent recruitment to the site of injury.

A skilled artisan would appreciate the term “punch biopsy” as primary technique to obtain diagnostic, full-thickness skin specimens. It is performed using a circular blade or trephine attached to a pencil-like handle. The instrument is rotated down through the epidermis and dermis, and into the subcutaneous fat.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

In one embodiment, the composition comprises of 10-100×10⁶ mesenchymal stromal cells resuspended in buffered saline or another appropriate final resuspension medium. In another embodiment, the composition comprises of 10×10⁶ mesenchymal stromal cells resuspended in buffered saline or other appropriate final resuspension medium. In another embodiment, the composition comprises of 15×10⁶ mesenchymal stromal cells resuspended in buffered saline or other appropriate final resuspension medium. In another embodiment, the composition comprises of 20-25×10⁶ mesenchymal stromal cells resuspended in buffered saline or other appropriate final resuspension medium. In another embodiment, the composition comprises of 20×10⁶ mesenchymal stromal cells resuspended in buffered saline or other appropriate final resuspension medium. In another embodiment, the composition comprises of 25×10⁶ mesenchymal stromal cells resuspended in buffered saline or other appropriate final resuspension medium. In another embodiment, the composition comprises of 30×10⁶ mesenchymal stromal cells resuspended in buffered saline or other appropriate final resuspension medium. In another embodiment, the composition comprises of 35×10⁶ mesenchymal stromal cells resuspended in buffered saline or other appropriate final resuspension medium. In another embodiment, the composition comprises of 40×10⁶ mesenchymal stromal cells resuspended in buffered saline or other appropriate final resuspension medium. In another embodiment, the composition comprises of 50×10⁶ mesenchymal stem cells resuspended in buffered saline or other appropriate final resuspension medium. In another embodiment, the composition comprises of 60×10⁶ mesenchymal stromal cells resuspended in buffered saline or other appropriate final resuspension medium. In another embodiment, the composition comprises of 70×10⁶ mesenchymal stromal cells resuspended in buffered saline or other appropriate final resuspension medium. In another embodiment, the composition comprises of 80×10⁶ mesenchymal stromal cells resuspended in buffered saline or other appropriate final resuspension medium. In another embodiment, the composition comprises of 90×10⁶ mesenchymal stromal cells resuspended in buffered saline or other appropriate final resuspension medium. In another embodiment, the composition comprises of 100×10⁶ mesenchymal stromal cells resuspended in buffered saline or other appropriate final resuspension medium.

In one embodiment, the final resuspension medium comprises Plasmalyte 148 pH 7.4 with the addition of serum proteins. In one embodiment, the proteins are present in concentrations of 2%-4%. In another embodiment, the final resuspension medium comprises human serum albumin or synthetic albumin Plasmalyte supplemented with 4% human serum albumin (HSA).

In one embodiment, the suspension medium presents reduced levels of lactate dehydrogenase (LDH) activity, reduced reactive oxygen species or combination thereof. In another embodiment, the suspension medium presents reduced levels of LDH activity. In another embodiment, the suspension medium presents reduced reactive oxygen species. In one embodiment, the reduced levels of LDH activity, reduced reactive oxygen species or combination thereof reflect reduced stress levels of cells and maintenance of an optimum metabolic state.

A skilled artisan would appreciate that lactate dehydrogenase (LDH) is a cytosolic enzyme present in many different cell types. LDH catalyzes the conversion of lactate to pyruvate and back, as it converts NAD+to NADH and back. LDH is released into the cell culture medium upon damage to the plasma membrane.

A skilled artisan would appreciate that increased reactive oxygen species (ROS) as highly reactive chemical molecules formed due to the electron receptivity of O₂. Examples of ROS include peroxides, superoxide, hydroxyl radical, singlet oxygen, and alpha-oxygen. Oxidative stress effects ex vivo culture expansion and longevity of MSCs, which has implications for cell therapy.

Cells can respond to stress in various ways ranging from the activation of survival pathways to the initiation of cell death that eventually eliminates damaged cells. Whether cells mount a protective or destructive stress response depends to a large extent on the nature and duration of the stress as well as the cell type. Also, there is often the interplay between these responses that ultimately determines the fate of the stressed cell.

Metabolic performance is greatly enhanced by enzymes, but their production, maintenance, and space requirements put a burden on cells, and the thrust to keep this burden low has a major impact on metabolic states. Regulation of metabolic enzymes plays a crucial role in the maintenance of metabolic homeostasis, and in the capacity of living systems to undergo physiological adaptation under multiple environmental conditions. Metabolic regulation is achieved through a complex interplay of transcriptional and post-transcriptional mechanisms, some of which have been experimentally characterized for specific pathways and organisms. In microenvironments featuring high tissue damage and oxidative stress, the therapeutic potential of hMSC significantly deteriorates, while apoptosis, autophagy and mitochondria disfunction is caused indirectly, affecting MSC fate.

In one embodiment, disclosed herein is the use of the autologous composition as described above, for treating a disease in the subject.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combinations thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

In one embodiment, the disease comprises arthritic lesion progression.

In one embodiment, the disease comprises osteoarthritis, multiple sclerosis, amyotrophic lateral sclerosis, degenerative disc disease, traumatic spinal cord injury, cardiovascular disease, atherosclerosis, ischemic myocardial tissue, chronic kidney disease, skin lesions, burns, ulceration, inflammatory lung disease or any combination thereof. In another embodiment, the disease comprises osteoarthritis. In another embodiment, the disease comprises multiple sclerosis. In another embodiment, the disease comprises amyotrophic lateral sclerosis. In another embodiment, the disease comprises degenerative disc disease. In another embodiment, the disease comprises traumatic spinal cord injury. In another embodiment, the disease comprises cardiovascular disease. In another embodiment, the disease comprises atherosclerosis. In another embodiment, the disease comprises ischemic myocardial tissue. In another embodiment, the disease comprises chronic kidney disease. In another embodiment, the disease comprises skin lesions. In another embodiment, the disease comprises burns. In another embodiment, the disease comprises ulceration. In another embodiment, the disease comprises inflammatory lung disease.

In one embodiment, the inflammatory lung disease comprises acute respiratory distress syndrome, pneumonia, asthma, chronic obstructive pulmonary disease, idiopathic pulmonary fibrosis or any combination thereof. In another embodiment, the inflammatory lung disease comprises acute respiratory distress syndrome. In another embodiment, the inflammatory lung disease comprises pneumonia. In another embodiment, the inflammatory lung disease comprises asthma. In another embodiment, the inflammatory lung disease comprises chronic obstructive pulmonary disease. In another embodiment, the inflammatory lung disease comprises idiopathic pulmonary fibrosis.

### Method for Preparation.

In some embodiments, disclosed herein is a method for the preparation of a composition comprising autologous mesenchymal stromal cells, comprising the step of collecting adipose tissue by punch biopsy.

In one embodiment, the cells are preserved in their natural anatomic compartment and tissue architecture is preserved up until isolation manipulations take place. In one embodiment, the cells are embedded in fat tissue in the hypoxic state that is needed for their optimal metabolic activity, up until isolation manipulation take place. In another embodiment, the biopsy is collected in a falcon containing 10 ml HBSS solution and the sample is processed in the lab within 24 h. In another embodiment, UW® cold storage solution is used and cold-chain (2-8 C) transport with ice packs is established.

In one embodiment, the tissue biopsy ranges from 0.5-2 g of tissue. In another embodiment, the tissue biopsy comprises 0.5 g of tissue. In another embodiment, the tissue biopsy comprises 0.6 g of tissue. In another embodiment, the tissue biopsy comprises 0.7 g of tissue. In another embodiment, the tissue biopsy comprises 0.8 g of tissue. In another embodiment, the tissue biopsy comprises 0.9 g of tissue. In another embodiment, the tissue biopsy comprises 1.0 g of tissue. In another embodiment, the tissue biopsy comprises 1.1 g of tissue. In another embodiment, the tissue biopsy comprises 1.2 g of tissue. In another embodiment, the tissue biopsy comprises 1.3 g of tissue. In another embodiment, the tissue biopsy comprises 1.4 g of tissue. In another embodiment, the tissue biopsy comprises 1.5 g of tissue. In another embodiment, the tissue biopsy comprises 1.6 g of tissue. In another embodiment, the tissue biopsy comprises 1.7 g of tissue. In another embodiment, the tissue biopsy comprises 1.8 g of tissue. In another embodiment, the tissue biopsy comprises 1.9 g of tissue. In another embodiment, the tissue biopsy comprises 2.0 g of tissue.

In one embodiment, the tissue biopsy is collected from subcutaneous fat.

In one embodiment, the tissue biopsy is obtained by a 0.5-1 cm skin incision. In another embodiment, the tissue biopsy is obtained by a 0.5 cm skin incision. In another embodiment, the tissue biopsy is obtained by a 0.6 cm skin incision. In another embodiment, the tissue biopsy is obtained by a 0.7 cm skin incision. In another embodiment, the tissue biopsy is obtained by a 0.8 cm skin incision. In another embodiment, the tissue biopsy is obtained by a 0.9 cm skin incision. In another embodiment, the tissue biopsy is obtained by a 1 cm skin incision.

In another embodiment, the biopsy is performed by a 6 mm punch biopsy device.

In another embodiment, the method for the preparation of a composition comprising autologous mesenchymal stromal cells further comprises: a. Isolating and culturing the adipose-derived stromal cells; b. Collecting the adipose-derived stromal cells; c. Expanding the mesenchymal stromal cells ex vivo in a Grade B clean room facility under Good Manufacturing Practices (GMP) operation; and d. Cryopreserving and thawing of cryopreserved adipose-derived stromal cells.

A skilled artisan would appreciate that Good Manufacturing Practices (GMP, also referred to as ‘cGMP’ or ‘current Good Manufacturing Practice’) is the aspect of quality assurance that ensures that medicinal products are consistently produced and controlled to the quality standards appropriate to their intended use and as required by the product specification, the Grade B cleanroom needs to meet a maximum of 3,520 particles (up to 0.5 μm diameter) per cubic foot.

### Method for Use.

In some embodiments, disclosed herein is a method for delaying osteoarthritic lesion progression, inducing repair and regeneration of inflamed/destructed tissue, or combination thereof, in a subject, comprising administering an autologous composition comprising mesenchymal stromal cells, wherein said mesenchymal stromal cells are derived from adipose tissue collected by punch biopsy from a subject, wherein said subject in need of delaying osteoarthritic lesion progression, inducing repair and regeneration of inflamed/destructed tissue or any combination thereof.

In one embodiment, the subject is diagnosed with osteoarthritis, multiple sclerosis, amyotrophic lateral sclerosis, degenerative disc disease, traumatic spinal cord injury, cardiovascular disease, atherosclerosis, ischemic myocardial tissue, chronic kidney disease, skin lesions, burns, ulceration, inflammatory lung disease of multiple causes or any combination thereof. In another embodiment, the subject is diagnosed with osteoarthritis. In another embodiment, the subject is diagnosed with multiple sclerosis. In another embodiment, the subject is diagnosed with amyotrophic lateral sclerosis. In another embodiment, the subject is diagnosed with degenerative disc disease. In another embodiment, the subject is diagnosed with traumatic spinal cord injury. In another embodiment, the subject is diagnosed with cardiovascular disease. In another embodiment, the subject is diagnosed with atherosclerosis. In another embodiment, the subject is diagnosed with ischemic myocardial tissue. In another embodiment, the subject is diagnosed with chronic kidney disease. In another embodiment, the subject is diagnosed with skin lesions. In another embodiment, the subject is diagnosed with burns. In another embodiment, the subject is diagnosed with ulceration. In another embodiment, the subject is diagnosed with inflammatory lung disease.

In one embodiment, the inflammatory lung disease comprises acute respiratory distress syndrome, pneumonia, asthma, chronic obstructive pulmonary diseases, and idiopathic pulmonary fibrosis. In another embodiment, the inflammatory lung disease comprises acute respiratory distress syndrome. In another embodiment, the inflammatory lung disease comprises pneumonia. In another embodiment, the inflammatory lung disease comprises asthma. In another embodiment, the inflammatory lung disease comprises chronic obstructive pulmonary disease. In another embodiment, the inflammatory lung disease comprises idiopathic pulmonary fibrosis.

In one embodiment, the inflammatory lung disease results from viral infection.

In one embodiment, the viral infection comprises rhinoviruse, enteroviruse (Picornaviridae), influenza viruse (Orthomyxoviridae), parainfluenza, metapneumoviruse, respiratory syncytial viruse (Paramyxoviridae), coronaviruse (Coronaviridae), adenoviruse, or any combination thereof. In another embodiment, the viral infection comprises rhinoviruse. In another embodiment, the viral infection comprises enteroviruse (Picornaviridae). In another embodiment, the viral infection comprises influenza viruse (Orthomyxoviridae). In another embodiment, the viral infection comprises parainfluenza. In another embodiment, the viral infection comprises metapneumoviruse. In another embodiment, the viral infection comprises respiratory syncytial viruse (Paramyxoviridae). In another embodiment, the viral infection comprises coronaviruse (Coronaviridae). In another embodiment, the viral infection comprises adenoviruse.

In one embodiment, the corona viruses comprise COVID-19.

A skilled artisan will recognize that Covid-19, also termed “novel coronavirus pneumonia”, “NCP”, “SARS-COV-2 acute respiratory disease”, and “COVID-19” comprises an infectious respiratory disease caused by the 2019 novel coronavirus (SARS-CoV-2), which was first detected during the 2019-20 Wuhan coronavirus outbreak. In some embodiments, SARS-COV-2 is transmitted through human-to-human transmission, generally via respiratory droplets as sneeze, cough or exhalation. In some embodiments, NCP symptoms appear after an incubation period of between 2 to 14 days. In some embodiments, coronavirus primarily affects the lower respiratory tract. In some embodiments, coronavirus primarily affects the upper respiratory tract. In some embodiments, NCP symptoms comprise fever, coughing, shortness of breath, pain in the muscles, tiredness, pneumonia, acute respiratory distress syndrome, sepsis, septic shock, death, or any combination thereof.

A skilled artisan will recognize that seven coronavirus types are known to affect humans. The compositions and methods disclosed herein are useful for treating any of them. In some embodiments, the coronavirus comprises Human coronavirus 229E (HCoV-229E). In some embodiments, the coronavirus comprises Human coronavirus OC43 (HCoV-OC43). In some embodiments, the coronavirus comprises severe acute respiratory syndrome-related coronavirus (SARS-COV). In some embodiments, the coronavirus comprises Human coronavirus NL63 (HCoV-NL63, New Haven coronavirus). In some embodiments, the coronavirus comprises Human coronavirus HKU1. In some embodiments, the coronavirus comprises Middle East respiratory syndrome-related coronavirus (MERS-COV), previously known as novel coronavirus 2012 and HCoV-EMC. In some embodiments, the coronavirus comprises Novel coronavirus (SARS-COV-2), also known as Wuhan coronavirus.

In one embodiment, the autologous composition is administered intra-articularly, systemically, intra-vascularly, intra-cerebroventricularly, sub-cutaneously, intra-dermally, intrathecally or intra-myocardially. In another embodiment, the composition is administered intra-articularly. In another embodiment, the composition is administered systemically. In another embodiment, the composition is administered intra-vascularly. In another embodiment, the composition is administered intra-cerebroventricularly. In another embodiment, the composition is administered sub-cutaneously. In another embodiment, the composition is administered intra-dermally. In another embodiment, the composition is administered intrathecally. In another embodiment, the composition is administered intra-myocardially.

### EXAMPLES

### Example 1—Identification of Potential Metabolic Differences in Mesenchymal Stromal Cells (MSC)

The purpose of this experiment was to identify potential metabolic differences in mesenchymal stromal cells (MSC) derived from adipose tissue using two different methods: comparison of metabolic state of cells obtained from liposuction (lipoaspirate) versus intact fat tissue (punch biopsy).

In order to compare metabolic parameters between lipoaspirate (LA) and intact fat tissue biopsy (FB) samples from the same patient were used, to essentially nullify any confounding factors relating to inter-individual differences such as genotype, age, health status etc. Testing samples obtained the same day from the same patient allowed the most optimal comparison conditions possible, as differences observed in samples from different patients cannot be possibly accounted for the 2 distinct tissue collection methods but can definitely arise due to multifactorial differences in the aforementioned parameters.

For each patient, the surgeon was provided with two vials (50 ml falcon tubes) filled with 25 ml HBSS solution, an isotonic saline buffer, which serves as the transportation liquid medium for the live tissues until they reach the lab, within 24 h post-operation at ambient temperatures (no cold chain transport).

The vials were disinfected externally and entered in the clean room where the processing took place. The liquid was separated from the tissue using Steriflip filters and the volume of liquid was measured and verified to be similar starting volume for both LA and FB. The wet mass of fat tissue from both samples was measured.

Volume of the liquid was usually similar (differences of up to 10% are not taken into account) but the mass was a lot different as a lot more material for the LA sample than the FB sample was obtained. This was documented to serve as a normalization factor since LDH secretion, a sign of tissue damage, is analogous to the amount of cells/tissue.

The initial liquid that was retrieved from each of the two samples was termed as the incoming liquid samples. The tissues had been kept in this liquid from the time of acquisition from the patient's body until the separation and further processing in the lab, so during this time and according to the tissue damage from each sample, LDH was being secreted in the surrounding liquid. This was what was measured directly, LDH concentration in the incoming liquid volume. Major producers of LDH are the adipocytes themselves, which are particularly sensitive to mechanical damage and if disrupted, they release LDH in the solution. To measure LDH concentration in the solution the Lactate Dehydrogenase Activity Assay Kit (Product Nr MAK066) from Sigma-Aldrich was used according to manufacturer's instructions.

Besides the collection and analysis of the incoming liquid sample, the inventors proceed with downstream processing of the tissue with collagenase to digest connective tissue and extracellular material and release the crude mix of cells which are collected after washes and centrifugations.

To this end, an equal amount of tissue mass from each sample (1 gr of LA and 1 gr of FB) was digested with collagenase in separate tubes. After the last washing step and centrifugation, a pellet of crude cells was collected. Importantly, the resulting pellet contains different kinds of cells that are present in the tissue, not only MSCs, so all stromal cells termed as stromal vascular fraction (SVF), including MSCs, endothelial cells, fibroblasts but also the ubiquitous Red Blood Cells (RBCs). This means that measurements of oxidative stress levels in such a sample are not derived solely from MSCs but reflect the total cumulative oxidative stress status of this first crude cell isolate.

Measurement of Intracellular Reactive Oxygen Species (ROS) in the stromal vascular fraction:
ROS-sensitive DCF-DA (2′,7′-dichlorofluorescin diacetate; Sigma Aldrich; 20 μM final concentration) was added in isolated SVF (100 μl).
Cells were incubated for 15 minutes at 37° C.
Cells were then washed with PBS, centrifuged for 5 min at 300 g and resuspended in 100 μl PBS.
For quantification of ROS generation, the fluorescence of DCF was measured using Arthur™ Image Based Cell Analyzer (NanoEntek).

The filters used for fluorescence measurement were Ex 466/40 and Em 525/50. The same threshold and acquisition parameters were used for both samples. Quantitative values of mean fluorescent intensity (expressed in arbitrary units) were digitally extracted and analyzed. The fluorescent intensity had a positive linear correlation with ROS concentration. The more oxidative stress, in other words, the more intense green fluorescence was emitted by the sample and recorded by machine.

### Results.

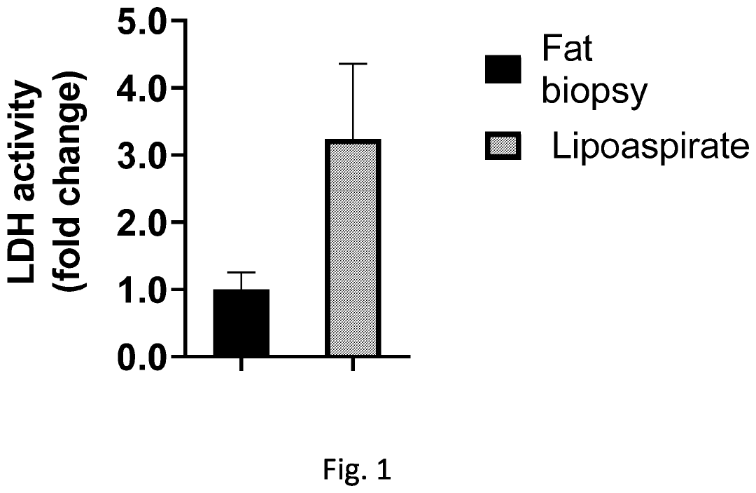

After the appropriate adjustments to account for the difference in collected mass of tissue, the incoming liquid sample was assayed for the presence of LDH, a marker of tissue damage, using the LDH kit described previously. Results are shown in FIG. 1.

As can be seen, more LDH is detected in the Lipoaspirate sample compared to the Fat Biopsy sample, both from the same subject. This means, if further verified with more samples, that adipose tissue obtained using lipoaspiration is more mechanically stressed than adipose tissue obtained with the punch biopsy method.

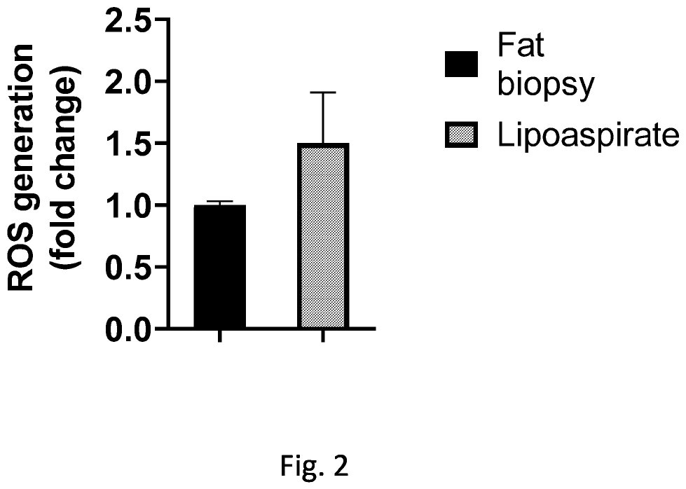

Next, the ROS levels were measured. Here again, higher ROS levels were quantified for the LA samples in relation to the FB samples (FIG. 2). As can be seen, Higher relative values for mean fluorescence intensity are detected for the LA samples, 1.5 times up to 2-fold increase.

### Conclusions.

A metabolic difference in the status of the Lipoaspirate relative to the Fat Biopsy was detected, in the incoming sample stage. The LA samples showed more tissue damage stress (increased LDH) and higher oxidative stress (ROS levels) than the intact FB sample.

### Example 2—Comparison of the Lipoaspirate Secretome with the Punch Biopsy

Adipose-derived human adult mesenchymal stromal cells (AD-MSCs) derived from Lipoaspirate and Punch Biopsy are cultured in parallel. Each culture is split into 2 T25 flasks, one that will be left untreated (baseline control) and one that gets treated with 10 μg/ml INFγ (stimulation) in serum-free medium (Nutristem). 3 days post-stimulation the culture supernatant (conditioned medium) is collected (5 ml volume). The samples are sent to the Proteomics facility of BSRC Fleming institute (Vari, Greece) for proteomics analysis using LC-MS/MS (Mass Spectrometry). The data analysis provides the differentially expressed proteins and the protein signature of the secretome, comparing lipoaspirate AD-MSCs and biopsy AD-MSCs. Since there will be subculturing of the cells in identical conditions for this stage, differences in the secreted proteins are not detected.

### Example 3—Adjustments of the Starting Biopsy Material

The advantage of a minimal starting biopsy material is that the patient no longer needs to undergo operation under anesthesia with its inherent risks and side-effects in a hospital setting to obtain lipoaspirate (plus the unpleasantly painful recovery from the procedure). A simple incision with a 6 mm punch biopsy tool that can be performed at the doctor's office under local anesthesia and subsequent removal of a small amount of subcutaneous adipose tissue of ~1 g is more than enough to get a PO culture going. Previous results show that processing of even 100 mg cleared adipose tissue for extracting SVF and culture MSCs. Such cultures typically took more than 1 month to reach ≥20 million cultured stromal cells and only very rarely resulted in rejected/aborted cultures that failed to grow. Often the lag phase of the initial PO cell culture from a tiny biopsy was rather prolonged.

However, as the amount of starting material is a crucial predictive parameter for the trajectory of a stromal cell culture (bigger biopsy-more cells as starting material-faster kinetics overall), it was decided to set a minimum limit in the acceptance criteria of the incoming material, from which the Drug Substance (DS), the plastic adherent cells, are extracted. The main bottleneck in the growth kinetics of a culture is the initial lag phase of the growth curve and this is proportionally related to the amount of the initial adipose tissue that undergoes processing. By setting a minimum of 1 g as the net weight of the tissue to be processed, the success of initial PO seeding and the rapid growth kinetics of the culture are ensured. This increases the predictability of the growth kinetics and the cell culture behavior since the risk factor of having a too sparse starting seeding density is excluded. For instance, a culture emanating from 1 gr of tissue can reach 70% confluency in a T25 within 5 days, while starting with 250 mg it can take even 1.5 weeks to reach the same confluency.

| Sample | Weight (gr) | Total days in P0 | Cell yield | Culture medium |
|---|---|---|---|---|
| 7919 | 0.448 | 8 | N/A | R&D |
| 7914 | 0.417 | 8 | N/A | R&D |
| 7917 | 1.140 | 5 | N/A | R&D |
| 7897 | 0.457 | 8 | N/A | R&D |
| 7918 | 0.406 | 9 | N/A | R&D |
| 7877 | 2.000 | 6 | 470000 | R&D |
| 7840 | 0.439 | 7 | 125000 | R&D |
| 7866 | 0.480 | 10 | N/A | R&D |
| 7935 | 0.984 | 6 | 405000 | R&D |
| 7874 | 0.928 | 8 | 515000 | R&D |
| 7940 | 1.293 | 8 | 1000000 | R&D |
| 7875 | 0.780 | 8 | 170000 | R&D |
| 7873 | 0.894 | 8 | 201500 | R&D |
| 7938 | 0.500 | 13 | 182500 | R&D |
| 7937 | 1.170 | 4 | 325000 | GMP |
| 7941 | 0.940 | 8 | 3180000 | GMP |
| 7954 | 0.200 | 13 | 2320000 | GMP |
| 7916 | 1.207 | 6 | 3525000 | GMP |
| 7955 | 1.000 | 7 | 450000 | GMP |
| 7957 | 0.511 | 8 | 150000 | GMP |
| 7957 | 1.527 | 8 | 487500 | GMP |
| 7932 | 1.600 | 8 | 5240000 | GMP |

The GMP-process comprises the use of Nutristem+5% human platelet lysate (hPL) and the R&D process comprises the use of DMEM+10% FBS.

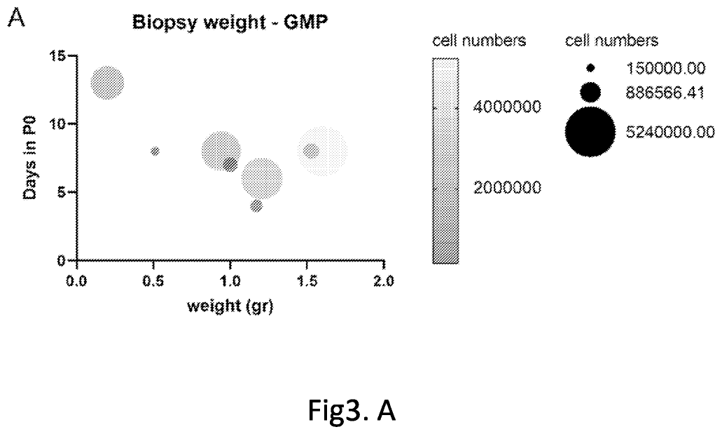

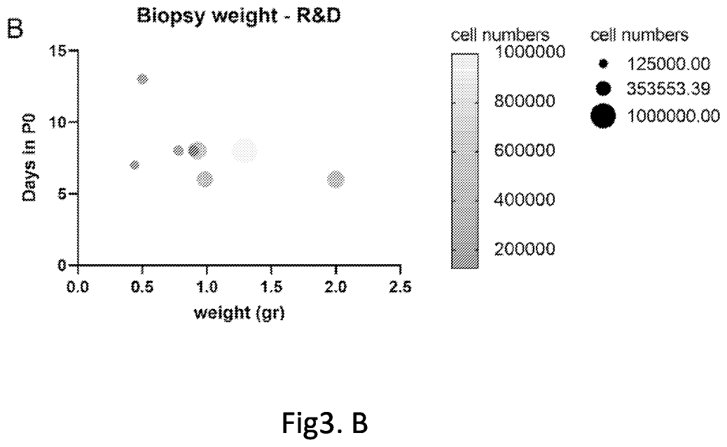

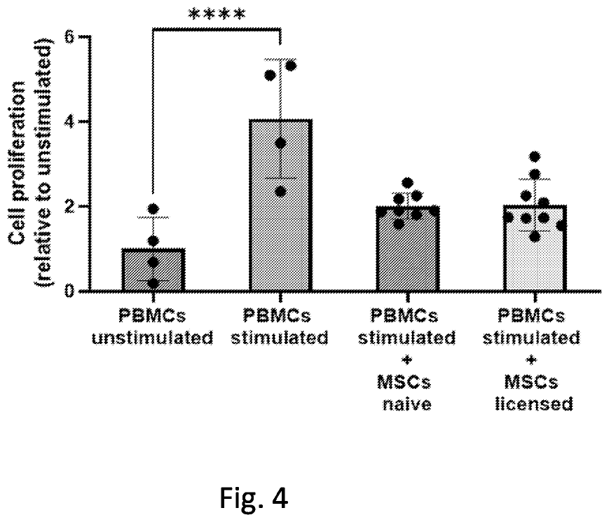

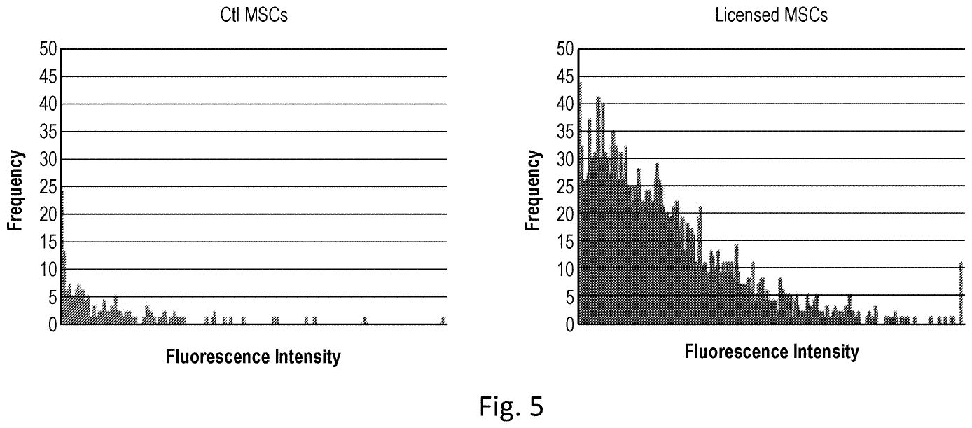

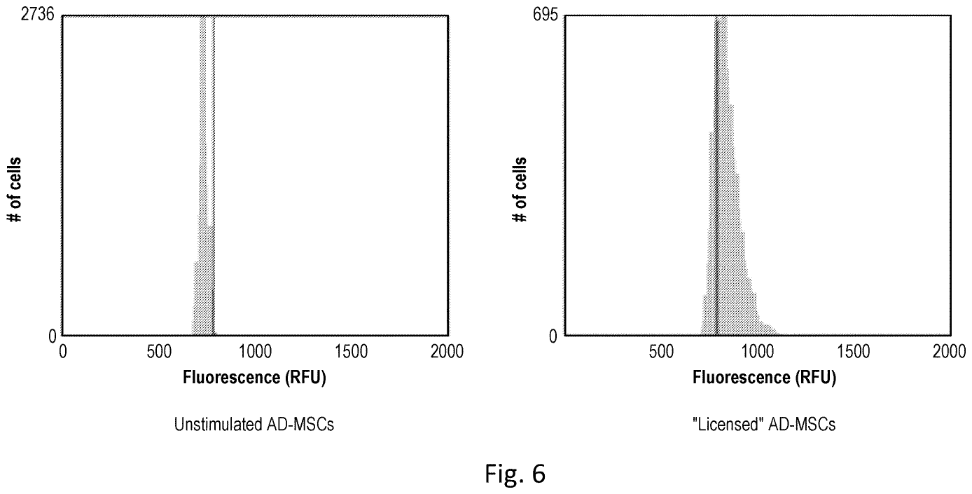

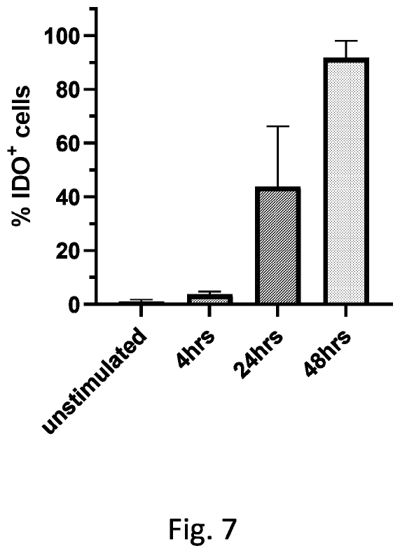

FIGS. 3A and 3B represent the amount of starting biopsy weight against the time to complete the PO stage and the total viable cell numbers collected at the end.

### Example 4—Immunosuppression on Activated PBMCs by MSCs—Potency Assay

Mesenchymal stem cells (MSCs) are thought to be a promising therapeutic agent due to their multiple paracrine and immunomodulatory properties, providing protection from chronic inflammation and promoting tissue repair. MSCs get stimulated in the inflammatory environment in the joint. Therefore, in this example, the paracrine MSC-mediated immunosuppression effect on proliferation of the activated peripheral blood mononuclear cells (PBMCs) was examined.

MSC mediated immunosuppression was analyzed using a PBMC proliferation assay. Primed MSCs (after INFγ-TNFα stimulation for 24 h) were seeded in the bottom chamber of a Transwell system with 0.4 μM pore size as to only allow soluble factors and extracellular vehicles to pass and be available to PBMCs.

A skilled artisan would understand the term “Transwell conditions” as co-culture plate with wells that have inserts. One cell population is seeded in the wells while the other is seeded in the inserts. These two populations share the same liquid and communicate by soluble molecules but do not come in physical contact as there is a membrane barrier, that lets substances and vesicles diffuse but does not allow cells to pass.

PBMCs were induced to proliferate using the ImmunoCult™ Human CD3/CD28 T Cell Activator (STEMCELL Technologies) and IL-2. ImmunoCult™ Human CD3/CD28 T Cell Activator is designed to activate and expand human T cells in the absence of magnetic beads, feeder cells, or antigen. ImmunoCult™ Human CD3/CD28 T Cell Activator consists of soluble antibody complexes that bind CD3 and CD28 cell surface ligands. Binding of the antibody complexes results in the cross-linking of CD3 and CD28 cell surface ligands, thereby providing the required primary and co-stimulatory signals for T cell activation. IL-2 acts primarily as a T cell growth factor, essential for the proliferation and survival of T cells. Untreated PBMCs were used as non-activated controls.

Although naïve MSCs are capable of suppressing T cell proliferation, the suppression potential varies among in vitro expanded cells and shows donor-dependent differences. MSCs can be “licensed” by inflammatory cytokines such IFN-γ and TNF-α to become more immunosuppressive and show a more homogeneous phenotype. For this assay, we evaluated the immunosuppressive effect of both naïve and licensed MSCs.

The relative proliferation rate, which was used as the basis for comparing the extent of suppression among the groups, was the Cell Counting Kit-8 (CCK-8; Dojindo Molecular Technologies, Inc., Kumamoto, Japan) absorbance ratios of the co-cultures with licensed or naïve MSCs. The CCK-8 method was performed according to the manufacturer's protocol. Cell Counting Assay (CCK-8) provides a convenient and robust way of performing a cell viability assay. The use of a water-soluble tetrazolium salt enables the quantification of the number of live cells by producing an orange formazan dye upon bio-reduction in the presence of an electron carrier.

The stimulation of PBMCs with CD3/CD28 and IL-2 resulted in activation of their proliferation, which was significantly reduced in coculture with naïve and licensed MSCs. In the experiments in which IFN-γ and TNF-α were used to activate MSCs, the cytokines were washed off before the addition of PBMCs.

### Example 5—Expression of Indoleamine Deoxygenase (IDO) Enzyme from Activated (Licensed) MSCs—Biological Test/Potency Assay

The purpose of this example was to show that the cells demonstrably have the ability to respond to a stimulus and show a behavior that is in line with the purported mechanism/mode of action. In the case of AD-MSCs for the treatment of Osteoarthritis (OA), the mode of action is suggested to be immunomodulation, a cardinal feature of MSCs in a variety of contexts. In OA there is a state of chronic inflammation in the synovium and the immunomodulation effect of MSCs acts to counteract this and dampen the inflammation. This is achieved by their quick response/stimulation by pro-inflammatory factors (eg TNFα or INFγ) which drives them to acquire a so-called “licensed” phenotype and secrete a plethora of factors or upregulate surface receptors that end up keeping proliferation and activation of leukocytes, macrophages and other immune cells in check. One of the major factors that get upregulated in licensed MSCs (INFγ-stimulated) and plays a cardinal role in immunomodulation, is the enzyme indoleamine 2,3-deoxigenase (IDO).

This example attempted to setup a potency assay based on the intracellular detection of IDO. The rationale is that MSCs that are able to respond to INFγ by producing IDO are in a position to negatively regulate the proliferation of lymphocytes and thus achieve immunomodulation. Since unstimulated naïve MSCs do not produce IDO, a simple release test would be to accept as relatively potent (relative and not absolute potency assay), the cell batches that demonstrably turn on the expression of IDO after a stimulation pulse of up to 24 h with INFγ.

### Method of IDO Staining and Readout

IDO is an enzyme inside the cells that is detected by intracellular antibody staining using a fluorescently-labelled antibody. The following procedure was followed at the time of cell harvesting, as a biological test of the final product. As a pre-requisite, the stimulation took place 24 h before harvesting, using 10 ng/ml INFγ in the sister sentinel flask, meaning the flask that has been seeded with the same cell batch at the same time and density, from the same cell suspension as the seeding of the 2-Layer Cell Factory that contains the final product. The cells in the Cell Factory that are destined to form the final product are left unstimulated, to serve as the naive control in the IDO assay. Only the cells that are in the sister sentinel flask get INFγ stimulation to serve as the positive control. The AD-MSCs from the cell factory will encounter INFγ in the inflamed joint post-injection, so they will be equivalent to the ones in the sentinel flask that get stimulated.

### Reagents:

PBS
Cell staining buffer: Add 1% FBS to PBS.
Fixation buffer: 4% paraformaldehyde in PBS.
Permeabilization buffer: 0.1% saponin in cell staining buffer.

### Procedure:

Trypsinize cells to collect them or thaw cryopreserved cells.
Centrifuge at 300×g for 5 minutes and wash once with 1 ml PBS.
Gently add 1 mL fixation solution per 1×10⁶ cells and incubate for 15 minutes at room temperature.
Centrifuge at 300×g for 5 minutes at room temperature and remove the fixation buffer.
Wash fixed cells with cell staining buffer. Centrifuge at 300×g for 5 minutes and discard the supernatant.
Resuspend fixed cells in 1 mL permeabilization buffer and incubate 15 min at RT with shaking or mild pipetting (ideally a rotating platform).
Centrifuge at 400×g for 5 minutes at room temperature. Remove the supernatant.
Dilute cells in 100 μl permeabilization buffer. Add 4 μL Human IDO Phycoerythrin Monoclonal Ab/106 cells (R&D Systems #IC6030P).
Incubate for 25 to 30 minutes at 4° C. in the dark.
Add 2 mL permeabilization buffer and centrifuge at 400×g for 5 minutes at 4° C. Remove the supernatant and repeat.
Remove wash and resuspend in 60 μl PBS, mix well, gently.
Proceed with loading the cartridge (25 μl) for measuring using the Arthur device.
Acquire the measurements using the Red Assay settings of the fluorescent cell counter.

### Results of IDO Assay—Biological Release Test for CARTIL-S

CARTIL-S is an autologous cell product which consists of adipose-derived human adult mesenchymal stromal cells (AD-MSCs) for use in the treatment of Osteoarthritis (OA) by intraarticular injection. It was demonstrated that upon stimulation of AD-MSCs with INFγ expression of IDO gets turned on and leads to a robust fluorescent detection signal after staining intracellularly with the PE-labelled IDO antibody.

### Example 6—Culture of Final Product CARTIL-S Combined with Fibrin Glue (Tisseel) Scaffold

Delivery of MSCs at specific lesions where the cells are needed to exert their local immunomodulatory and regenerative actions is a challenge, since it is known that MSCs in a buffer suspension can quickly disappear away from the site of injection. Strategies to counteract this are based on combining the cells with biocompatible 3D-scaffolds that provide a niche 3D-structure for the cells to adhere to and presumably get better retained locally.

The fibrin glue (marketed by Takeda/Baxter as Tisseel) is a 3D-scaffold which consists of a dual syringe with thrombin solution as component A in one chamber and sealer protein-aprotinin solution as component B in the second chamber. Cells get resuspended in the thrombin solution and using a Duplotip, a syringe tip that allows the two solutions to come into contact only at the very tip of the syringe in the outside environment, the clotting reaction starts. In a matter of a few seconds, the mixed solution solidifies and forms a porous clot. Cells become embedded in the clot and are thus attached in a proteinaceous 3D matrix.

The purpose of this example was to examine the AD-MSC behavior in 3D-scaffold environment using the fibrin glue (Tisseel, Baxter) as scaffold combined with the MSC Orthopedics Final Product dose which consists of 20 million cells at 10 million per ml concentration. Since this is the final product that is destined for application intra-particularly, it is desired to follow-up in vitro what happens to the cells when they are incubated with culture medium while embedded in the Tisseel fibrin glue and to observe scaffold stability in culture as well as potential cell differentiation.

AD-MSCs were cultured and harvested at Passage 2 from a 2-Layer Cell Factory according to the Batch Record protocol for CARTIL-S. Cells were counted, washed and subsequently divided into 2 samples of at least 20 million cells each. One sample was mixed with the Tisseel glue. A Duplotip was applied to the syringe, which has two canals and only allows the mixing of the 2 components at the very tip of the needle. Using the special pestle provided with the Tisseel syringe, 10 wells of a 12-well plate received 1/10th of the content as a small blob of fibrin glue. Keeping the pressure and amount as steady as possible, this account for ~200 microliters of fibrin glue with 2 million cells (of the total 20 million) in each well of the 12-well plate.

The fibrin glue with the cells started to solidify immediately, forming an amorphous whitish clot. Following solidification, culture medium was gently added to the wells and the plates were transferred to an incubator for culture at 37° C./5% CO₂/saturating humidity.

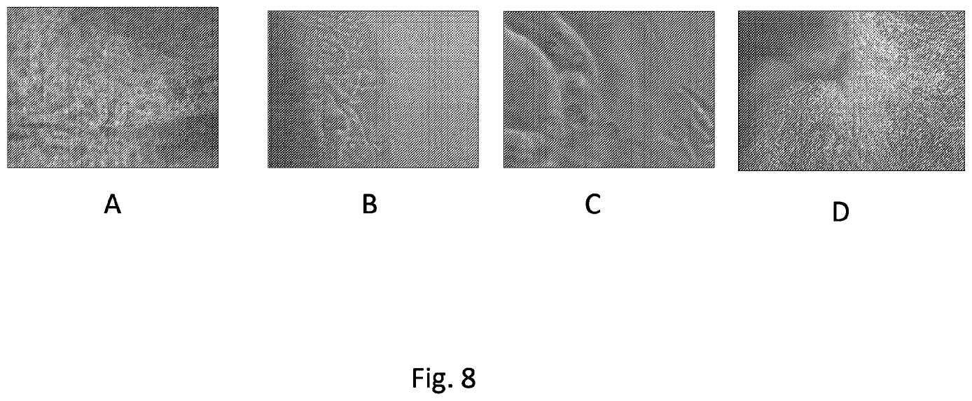

FIGS. 8A-D demonstrates the cells right seeded in the fibrin glue. The 3D scaffold did not show signs of degradation under the culture conditions of the incubator and was stable.

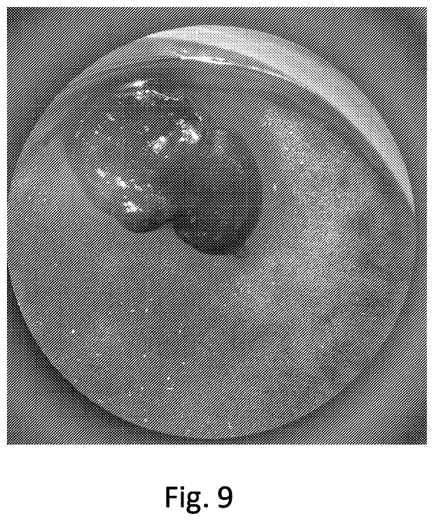

Next, a fixation and Alizarin Red staining protocol in the remaining wells followed, to visualize calcium deposits and bone formation (osteogenesis). Extracellular calcium deposits, a characteristic product of osteoblasts, stain intensely red with Alizarin Red dye. It was observed even macroscopically that the fibrin glued scaffold stains very intensely with this dye. A control of unpopulated cell-free scaffold alone is missing in this pilot experiment, and it may be that the scaffold itself absorbs the dye, nevertheless the result was a very intense red color especially on the scaffold but also on the rest of the plastic well. FIG. 9 is an image shot with a smartphone through the ocular of a stereoscope, providing the macroscopic view of a fibrin clot stained intensely with Alizarin Red.

From the above-described pilot experiment the following conclusions can be drawn:
AD-MSCs attach and proliferate in the fibrin glue scaffold, tolerate it very well.
The cells propagate out of the scaffold and quickly cover the whole surface.
The fibrin glue scaffold (Tisseel) is stable and does not get degraded in vitro (at least for up to 21 days of observation).
Trypsin treatment does not degrade the scaffold.
Successful osteogenic differentiation happens in and around the fibrin glue scaffold, even without dedicated osteogenesis-driving medium. Control missing here, the background staining of the scaffold alone, without cells.
Tisseel is established in clinical practice as biocompatible sealant matrix and is a good candidate to combine with AD-MSCs for local application of high-density cell transplant.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An autologous composition comprising mesenchymal stromal cells, wherein said mesenchymal stromal cells are derived from adipose tissue collected by punch biopsy from a subject.

2. The autologous composition according to claim 1, wherein said composition comprises of 10-100×10⁶ mesenchymal stromal cells resuspended in buffered saline or other appropriate final resuspension medium.

3. The autologous composition according to claim 2, wherein said composition comprises of 20-25×10⁶ mesenchymal stromal cells resuspended in buffered saline or other appropriate final resuspension medium.

4. The autologous composition according to claim 3, wherein said buffered saline suspension medium presents reduced levels of Lactate Dehydrogenase (LDH) activity, reduced reactive oxygen species or combination thereof.

5. Use of an autologous composition according to claim 1, for treating a disease in said subject.

6. Use according to claim 5, wherein said disease comprises arthritic lesion progression.

7. Use according to claim 5, wherein said disease comprises osteoarthritis, multiple sclerosis, amyotrophic lateral sclerosis, degenerative disc disease, traumatic spinal cord injury, cardiovascular disease, atherosclerosis, ischemic myocardial tissue, chronic kidney disease, skin lesions, burns, ulceration, inflammatory lung disease or any combination thereof.

8. The use according to claim 7, wherein said inflammatory lung disease comprises acute respiratory distress syndrome, pneumonia, asthma, chronic obstructive pulmonary diseases, idiopathic pulmonary fibrosis or any combination thereof.

9. (canceled)

10. (canceled)

11. (canceled)

12. Use according to claim 5, wherein said disease comprises osteoarthritis.

13. A method for the preparation of a composition comprising autologous mesenchymal stromal cells, comprising the step of collecting adipose tissue by punch biopsy.

14. The method according to claim 13, wherein the tissue biopsy ranges from 0.5-2 g of tissue.

15. The method according to claims 13-14, wherein said tissue biopsy is collected from subcutaneous fat.

16. The method according to claim 13, wherein said tissue biopsy is obtained by a 0.5-1 cm skin incision.

17. The method according to claim 16, wherein said biopsy is performed by a 6 mm punch biopsy device.

18. A method according to claim 13, further comprising the steps of
a. Isolating and culturing the adipose-derived stromal cells;
b. Collecting the adipose-derived stromal cells;
c. Expanding the mesenchymal stromal cells ex vivo in a Grade B clean room facility under Good Manufacturing Practices (GMP) operation; and
d. Cryopreserving and thawing of cryopreserved adipose-derived stromal cells.

19. A method for delaying osteoarthritic lesion progression, inducing repair and regeneration of inflamed/destructed tissue, or combination thereof, in a subject, comprising administering an autologous composition comprising mesenchymal stromal cells, wherein said mesenchymal stromal cells are derived from adipose tissue collected by punch biopsy from a subject, wherein said subject in need of delaying osteoarthritic lesion progression, inducing repair and regeneration of inflamed/destructed tissue or any combination thereof.

20. The method according to claim 19, wherein said subject is diagnosed with osteoarthritis, multiple sclerosis, amyotrophic lateral sclerosis, degenerative disc disease, traumatic spinal cord injury, cardiovascular disease, atherosclerosis, ischemic myocardial tissue, chronic kidney disease, skin lesions, burns, ulceration, inflammatory lung disease or any combination thereof.

21. The method according to claim 20, wherein said inflammatory lung disease comprises acute respiratory distress syndrome, pneumonia, asthma, chronic obstructive pulmonary disease, and idiopathic pulmonary fibrosis.

22. (canceled)

23. (canceled)

24. (canceled)

25. The method according to claim 19, wherein said subject is diagnosed with osteoarthritis.

26. The method according to claim 19, wherein said autologous composition is administered intra-articularly, systemically, intra-vascularly, intra-cerebroventricularly, sub-cutaneously, intra-dermally, intrathecally or intra-myocardially.
